(19) United States
(12) Patent Application Publication
(10) Pub. No.: US 2009/0099267 A1
(43) Pub. Date: Apr. 16, 2009

(54) POLYMERS, COMPOSITIONS AND METHODS OF MAKING THE SAME

(75) Inventors: Rajesh Kumar, Groton, CT (US); Jayant Kumar, Westford, MA (US); Virinder Singh Parmar, Lowell, MA (US); Arthur C. Watterson, Nashua, NH (US)

Correspondence Address: FISH & RICHARDSON PC, P.O. BOX 1022, MINNEAPOLIS, MN, 55440-1022, US

(73) Assignee: University of Massachusetts, Boston, MA (US)

(21) Appl. No.: 11/915,566

(22) PCT Filed: May 26, 2006
(86) PCT No.: PCT/US06/20915

§ 371 Date: Sep. 25, 2008

Related U.S. Application Data
(60) Provisional application No. 60/685,472, filed on May 27, 2005.

(51) Int. Cl. A61K 47/30; C08G 65/40; C08G 65/42; C08G 63/06; H01L 31/042; C08L 71/10; C07C 69/773; H01M 6/16

(52) U.S. Cl. 514/772.3; 528/206; 528/219; 528/207; 525/450; 560/61; 429/188; 136/263; 435/119; 435/135

(57) ABSTRACT

Polymers having a main chain having both aromatic units and aliphatic units (with repeating heteroatoms) and a side chain macromonomer are described. Methods of making these polymers using enzymatic synthesis and the applications of these polymers are also described.

### CROSS REFERENCE TO RELATED APPLICATION

This application claims priority to U.S. Provisional Patent Application No. 60/685,472, filed on May 27, 2005, the disclosure of which is incorporated herein by reference in its entirety.

### TECHNICAL FIELD

This invention relates to polymers, and more particularly to polymeric electrolytes.

### BACKGROUND

Polymer electrolytes are polymers or polymer compositions that are electrically conductive. Polymer electrolytes have generated widespread interest as solid-state alternatives to liquid and crystalline electrolytes for device applications, such as batteries, electro-chromic displays, and photo-electrochemical cells. Polymer electrolytes generally have useful ionic conductivities and can eliminate problems of sealing and solvent leakage associated with liquid electrolytes. The ionic conductivity of these electrolytes is believed to arise from their rapid segmental motion combined with strong Lewis type acid-base interactions between a cation and a donor atom present in their structure. Consequently, it is expected that higher ionic mobilities occur in the amorphous rather than crystalline phases of such materials.

Current studies have focused on developing alternative polymers that retain their ability to solvate and conduct ions, but remain amorphous at room temperature. There has been a great need to develop readily processable conducting polymers and polyelectrolytes that have high ionic conductivity and stability.

### SUMMARY

The invention is based, at least in part, on the discovery of polymeric materials that possess excellent conductivity and stability. These polymers include a main chain having both aromatic units and aliphatic units (with repeating heteroatoms) and a side chain macromonomer. The invention is also based, at least in part, on the discovery of enzymatic syntheses (biocatalytic processes) to prepare these polymeric materials in an environmentally benign manner.

In general, the invention features new polymers, including so-called first generation polymers and second generation polymers. First generation polymers can have a structure shown in formulae I-III, below. Second generation polymers can have a structure shown in formulae V-VII. In both first and second generation polymers, each R can be hydrogen, alkyl, aryl, or arylalkyl; R′ can be OH or alkoxy; J can be arylene, heteroarylene, or alkylene; Y can be independently O, S, or NH; Z, Z₁, and Z₂ can be independently O, S, or NH; Q can be alkylene, alkenylene, alkynylene, or arylene; m can be an integer of 0-100; n can be an integer of 1-1000; n₁ can be an integer of 1-1000; n₂ can be an integer of 1-10,000, and v is 1, 2 or 3.

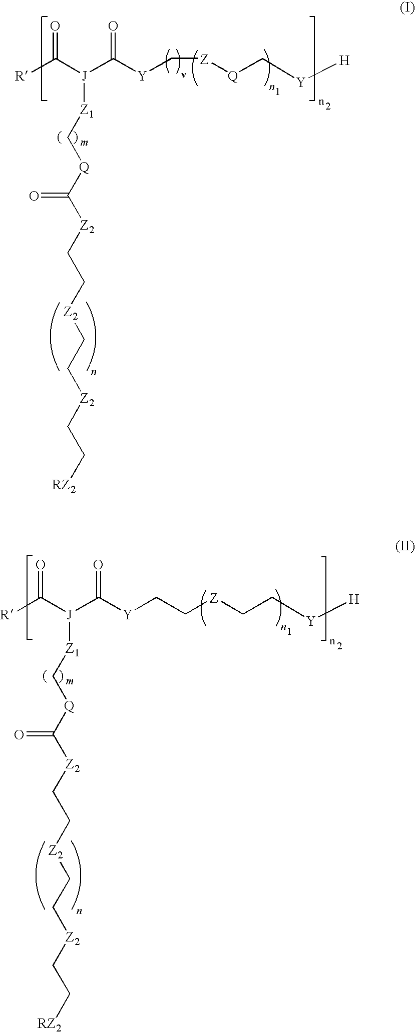

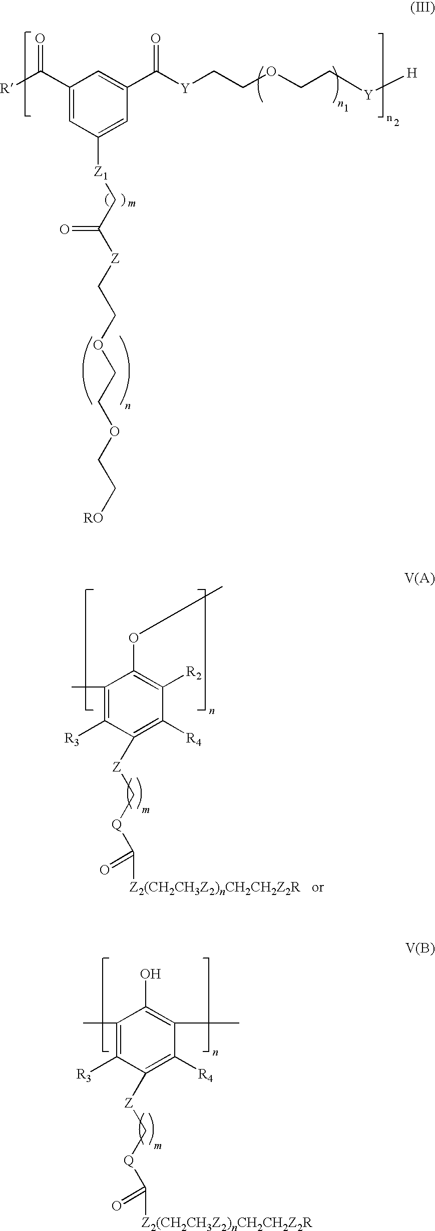

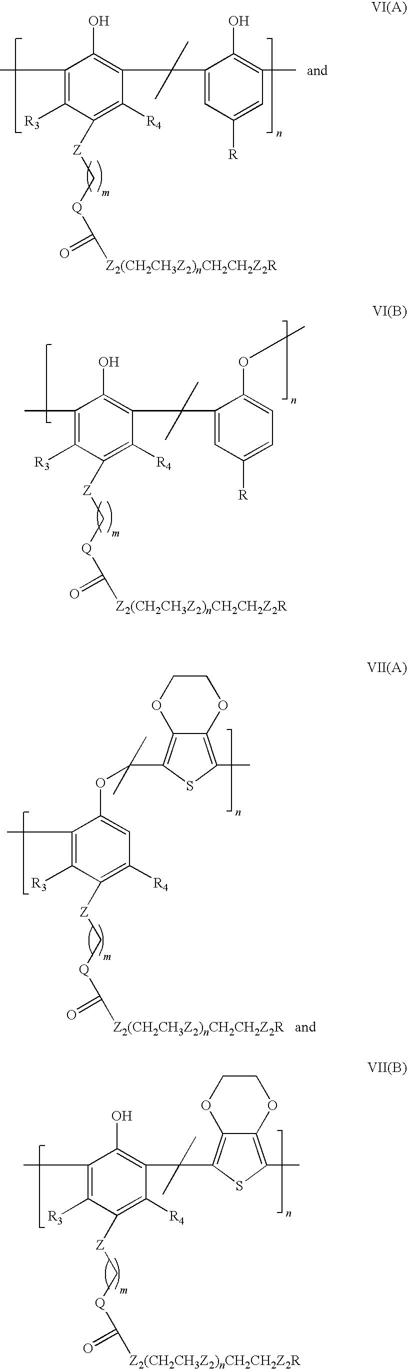

In one aspect, the invention features compositions including the new polymers I, II, III, V(A), V(B), VI(A), VI(B), VII(A), and/or VII(B). In another aspect, the invention features polymer electrolytes including new polymers I, II, III, V(A), V(B), VI(A), VI(B), VII(A), and/or VII(B), or mixtures thereof. In other aspects, the invention features polymer electrolytes including compositions containing the new polymers I, II, III, V(A), V(B), VI(A), VI(B), VII(A) and/or VII(B). In another aspect, the invention features solar cells and batteries including the new polymers I, II, III, V(A), V(B), VI(A), VI(B), VII(A), and/or VII(B), and solar cells including compositions containing the new polymers. The solar cells can further contain dyes, stabilizers, additives, etc. In another aspect, the invention features drug delivery agents including the new polymers or compositions containing the new polymers.

In another aspect, the invention features methods of preparing a macromonomer 3, by mixing a monomer or oligomer with an acyl compound to form a monomer mixture; adding a lipase, an esterase, or a protease to the monomer mixture to form a reaction mixture; and reacting the reaction mixture for a time and under conditions suitable to yield a macromonomer, wherein the macromonomer is an alkylene ester, alkylene amide, or an alkylene thioester. Macromonomer 3 has the following structure:

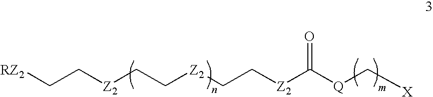

wherein R is hydrogen, alkyl, aryl, or arylalkyl; X is halide or sulfonate; Q is alkylene, alkenylene, alkynylene, or arylene; Z₂ is O, S, or NH; m is an integer of 1-100; and n is an integer of 1-1000.

In another aspect, the invention features methods of preparing first generation polymers (e.g., I, II, or III) by reacting a macromonomer with an arylene-based or heteroarylene-based polymer under suitable conditions to yield the polymeric electrolyte.

In another aspect, the invention features methods of preparing second generation polymers (e.g., V(A), V(B), VI(A), VI(B), VII(A), and/or VII(B)) by reacting a macromer with an arylene or heteroarylene under suitable enzymatic polymerization conditions in the presence of an oxidative enzyme, such as horse radish peroxidase.

The polymers prepared by the enzymatic processes described herein have the advantage of having greater stability compared to polymers that are prepared from acidic starting materials such as carboxylic acids. The enzymatic synthetic processes described herein have the added advantage of reducing or eliminating the use of chemical solvents and thereby significantly reducing environmental pollution caused by conventional chemical synthesis of polymers. Further, because of the chemical selectivity of enzymatic synthesis, the amount of reactants (i.e., monomers), which are required to complete a polymerization reaction and to produce a desired amount of a polymer product, can be precisely controlled to the right stoichiometry. In other words, no excess reactants are needed, which result in lower production costs for industrial manufacturing. Finally, because of the use of enzymes that require mild reaction conditions, the polymers that are prepared by the enzymatic synthesis described herein are generally biocompatible. There is also no template required for polymerization. As a result, these polymers can be used in a number of biomedical applications such as carriers for controlled drug delivery, tissue engineering, bio-implants, and scaffolds.

Unless otherwise defined, all technical and scientific terms used herein have the same meaning as commonly understood by one of ordinary skills in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the description, drawings, and from the claims.

### DETAILED DESCRIPTION

The polymers described herein include a main chain having both aromatic units and aliphatic units (with repeating heteroatoms) and a side chain macromonomer. The polymers can also include one or more other polymers, fillers, and/or additives.

The new polymers described herein include first generation polymers, shown in formulae I-III, and second generation polymers shown in formulae V-VII, in which each of R is hydrogen, alkyl, aryl, or arylalkyl; R′ is OH or alkoxy; J is arylene, heteroarylene, or alkylene; Y is independently O, S, or NH; Z, Z₁, and Z₂ are independently O, S, or NH; Q is alkylene, alkenylene, alkynylene, or arylene; m is an integer of 0-100; n is an integer of 1-1000; n₁ is an integer of 1-1000; n₂ is an integer of 1-10,000; and v is 1, 2, or 3.

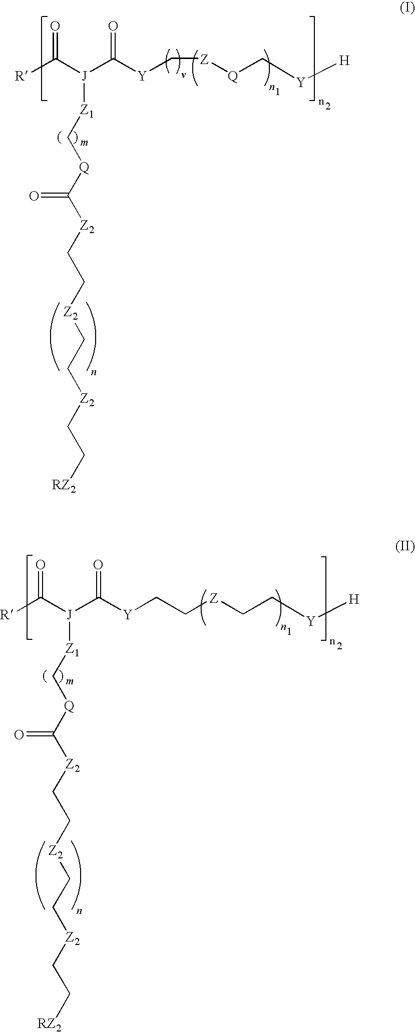

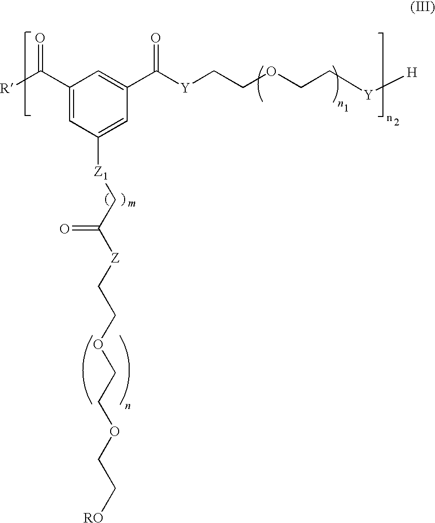

Alkyl groups used herein can include 1 to about 12 carbon atoms and are optionally unsubstituted or substituted. Examples of useful alkyl groups include methyl, ethyl, n-propyl, isopropyl, n-butyl, and n-hexyl.

Aryl groups used herein can include 5 to about 14 carbon atoms and are optionally unsubstituted or substituted. Examples of useful aryl groups include phenyl, naphthyl, tetrahydronaphthyl, indenyl, indanyl, anthracenyl, and fluorenyl ring systems.

Arylalkyl groups used herein can include alkyl groups substituted with aryl groups. Examples of useful arylalkyl groups include phenylmethyl, phenylethyl, and phenylpropyl.

Alkylene groups used herein can include 1 to about 12 carbon atoms and are optionally unsubstituted or substituted with heteroatoms, such as O, S, or NH. Examples of useful alkylene groups include methylene, ethylene, propylene, and butylenes.

Alkenylene and alkynylene groups used herein can include 2 to about 12 carbon atoms and are optionally unsubstituted or substituted with heteroatoms, wherein the heteroatoms are selected from O, S, or NH. Examples of useful alkenylene groups include ethenylene, propenylene, and butenylene. Examples of useful alkynylene groups include ethynylene, propynylene, and butynylene.

Arylene and heteroarylene groups used herein can include 5 to about 14 carbon atoms and are optionally unsubstituted or substituted. Examples of useful arylene groups include phenylene, naphthylene, indenylene, and anthracenylene. Examples of useful heteroarylene groups include pyrrolylene, pyrazolylene, imidazolylene, and oxazolylene.

The integer number m can be a number between 0-100 (e.g., 0, 1, 2, 5, 10, 20, 40, 50, 70, 80, 90, or 100). The integer number n can be a number between 1-1000 (e.g., 1, 2, 5, 10, 50, 100, 150, 200, 300, 400, 500, 900 or 1000). The integer number n₁ can be a number between 1-1000 (e.g., 1, 2, 5, 10, 50, 100, 150, 200, 300, 400, 500, 900 or 1000). The integer number n₂ can be a number between 1-10,000 (e.g., 1, 2, 5, 10, 50, 100, 500, 1000, 3000, 4000, 5000, 9000, or 10,000). The integer number v is 1, 2 or 3.

Electrolytes can be prepared from any of the polymers described herein, or blends of any of the polymers described herein, by adding an ionic compound, e.g., an ionic liquid such as 1,3-propylmethyl imidazolium iodide, to a solution, slurry or emulsion of the polymer or blend in a solvent, e.g., butyrolactone.

### Macromonomers

Macromonomers described herein include macromolecules that have one end-group that has a reactive functional group that enables it to act as a monomer molecule. The macromolecule can be linked to an aromatic unit in the main chain of the polymer. Macromonomers that can be used in the present invention include an aliphatic chain with repeating heteroatoms such as an alkylene, alkenylene, or alkynylene chain with repeating O, S, or NH groups with a terminal reactive functional group such as a halide, e.g., Cl, Br, or I, or a tosylate. Examples of polyethylene glycol and polyethylene diammine based macromonomers are shown below with a terminal bromide.

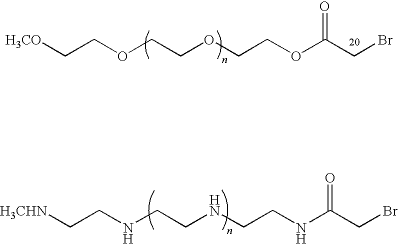

### Macromers

Macromers (e.g., macromer 6, below) are obtained by the reaction of macromonomers (e.g., PEG compounds with a leaving group) with hydroxy aryl or dihydroxy aryl compounds (e.g. dihydroquinone). Example of macromers are shown below.

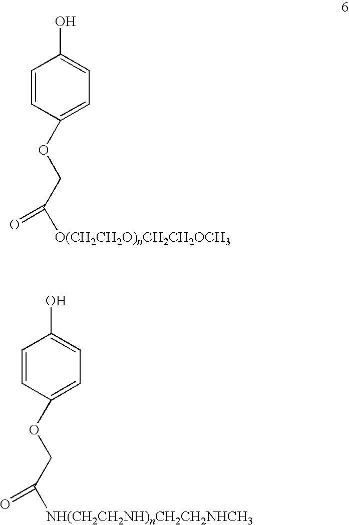

The first generation polymers (I, II, and III) are synthesized by reacting a macromonomer with an arylene-based or heteroarylene-based polymer under suitable conditions. The second generation polymers (e.g., V(A), V(B), VI(A), VI(B), VII(A) and/or VII(B)) are synthesized by reacting a macromer with an arylene or heteroarylene under suitable enzymatic polymerization conditions in the presence of an oxidative enzyme, such as a peroxidase, e.g., horse radish peroxidase, soybean peroxidase and Arthromyces ramosus peroxidase. The peroxidases can be unmodified or chemically modified, and are available from Sigma-Aldrich.

### Enzymatic Synthesis of Polymers

Synthesis of the Main Chain Polymer

Any enzymes that can catalyze the reactions that result in the new polymer products can be used in these methods. For instance, a lipase can be used in a polycondensation reaction that results in polymers such as polyesters (e.g., when Y is oxygen in formula (I)) and polyamides (e.g., when Y is amino in formula (I)). Similarly, esterases and proteases can also be used for polymerization. The enzymes can be either free in water or in liquid reactants, or immobilized, e.g., with agar gel, so that they can be recycled for repeated uses. The enzymes can be used either fresh after being isolated from culture or after being stored for an extended period of time so long as they remain active. The activity of the enzyme can be determined by using standard test kits available commercially or by using a substrate of the enzyme with a detectable product and by plotting the reaction kinetics.

Monomers that can be used to synthesize the main chain polymer include those that can undergo the new polymerization process, e.g., anhydrides, caprolactams, diols, diamines, and molecules that include polymerizable functionalities such as hydroxy, ester, thiol, thioester, and amino groups. Aryl and heteroaryl monomers can be used to synthesize the main chain polymer

To carry out the new methods, one can first mix monomers described herein and an enzyme suitable for the desired type of polymerization in a container or vessel, e.g., in a round bottom flask. The flask can then be placed in an oil or water bath maintained at a predetermined temperature (e.g., 10° C. to 120° C.) and the monomer mixture is stirred for a period of time. By-products can be removed by nitrogen flushing, azeotropic distillation, or vacuum. The enzyme can then be separated, e.g., by using water, and the product can then be purified by known methods. Lipases (e.g., Candida antarctica lipase, lipase A, and lipase B), and other enzymes such as esterases and proteases (e.g., papain and chymotrypsin), efficiently catalyze the polycondensation of various monomers as described herein, such as dimethyl 5-hydroxyisophthalate and polyethylene glycols, in a solvent-free system.

The new synthetic methods can be conducted under mild conditions that are suitable for enzymes. For instance, the reactions can be conducted at a temperature between 10° C. to 120° C. (e.g., 25, 50, 60, 70, 80, 90, 100, or 110° C.). The enzymatic synthesis can also be conducted in an environment free of organic solvents, e.g., in an aqueous solution or in a solvent-free condition.

Synthesis of the Macromonomers and the New Polymers

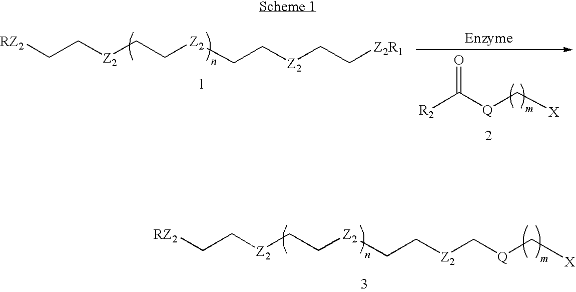

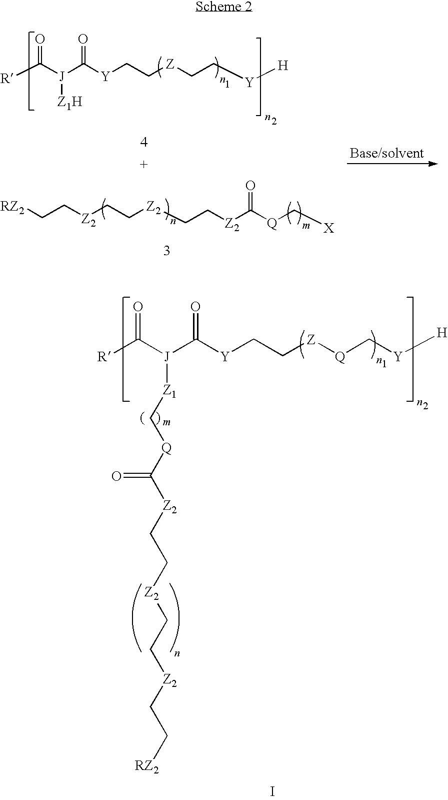

Schemes 1 and 2 are self-explanatory examples of enzymatic polymerization reactions that can be used to prepare the macromonomers and new polymers (polymers I, II, III, V(A), V(B), VI(A), VI(B), VII(A) and/or VII(B), or mixtures thereof), respectively. The definition of each of the variables (i.e., R, R′, J, Y, Z, Z₁, Z₂, Q, m, n, n₁, and n₂) is the same as that in formulae I-III.

Macromonomer 3 used herein can be synthesized using the enzymatic reaction shown in Scheme 1. An aliphatic chain with repeating heteroatoms 1 (e.g., polyethylene glycol) can be reacted with a acyl compound 2 (e.g., methyl chloroacetate) to prepare macromonomer 3 (e.g., chloromethyl ester of polyethylene glycol) in the presence of a suitable enzyme (e.g., Canidida antarctica lipase B).

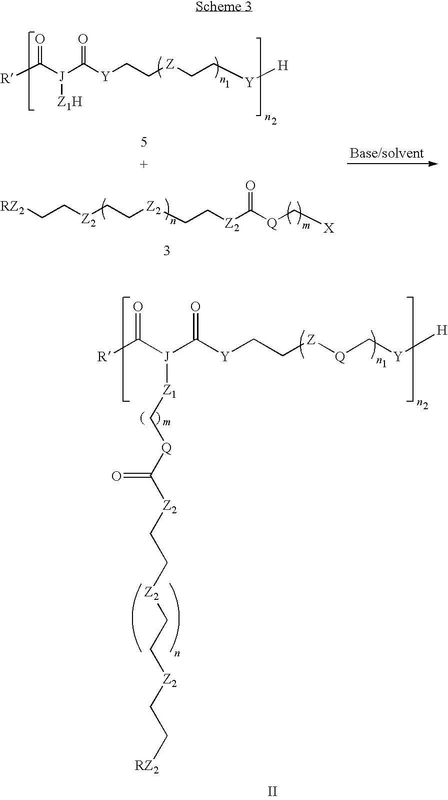

The new polymers described herein can be synthesized using the reactions shown in Schemes 2-8. Macromonomer 3 can be reacted with a main chain polymer 4, 5, and/or 6 to form new polymers I, II, and III respectively, as shown in reaction schemes 2-4. Similarly macromonomer 3 yields macromer IV as shown in reaction scheme 5. The reaction can be carried out using a suitable base and solvent to carry out the nucleophilic reaction between the macromonomer 3 and an arylene with nucleophilic groups (OH, NH₂ or SH) in the 1 and 4 position. The reaction temperature and time period may be determined by the reactants chosen to prepare new polymers 5. For instance, the reactions can be conducted at a temperature between 10° C. to 120° C. (e.g., 25, 50, 60, 70, 80, 90, 100, or 110° C.). Oxidative polymerization or peroxidase mediated polymerization yields new polymers V(A), V(B), VI(A), VI(B), VII(A) and VII(B) as shown in schemes 6-8. Polymers VI(A), VI(B), VII(A) and VII(B) are random copolymers, the slash on the bond between the two phenyl rings is a standard representation of random copolymers. Polymers VI(A) and VI(B) are obtained as a mixture of polymers. Similarly polymers VII(A) and VII(B) are also obtained as a mixture of polymers. R₁, R₂, R₃ and R₄ are independently hydrogen or alkyl groups. Z, Z₁, and Z₂ are independently O, S, or NH groups.

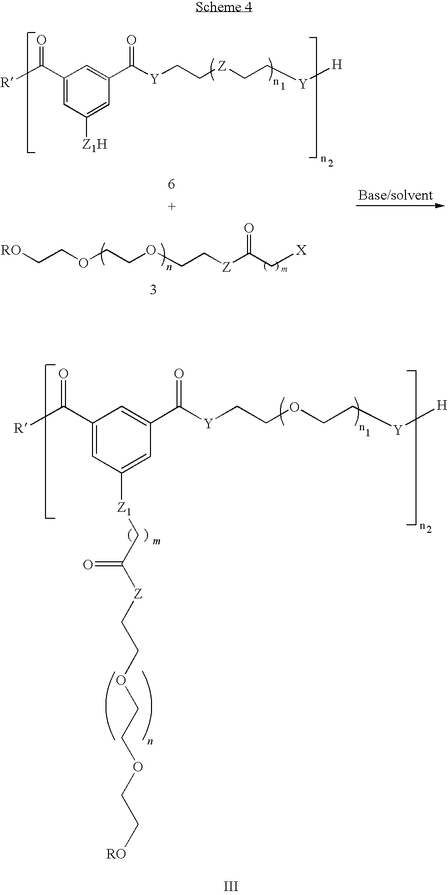

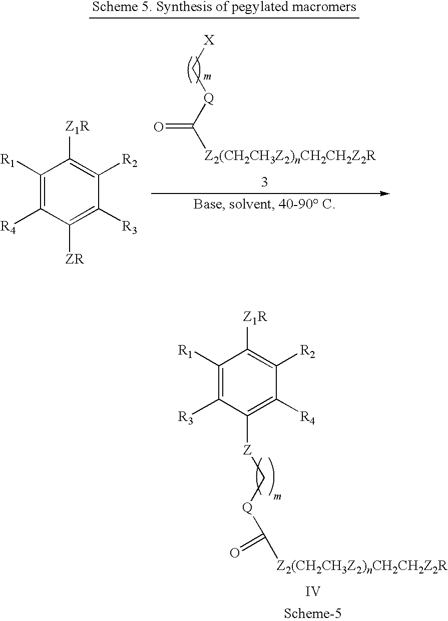

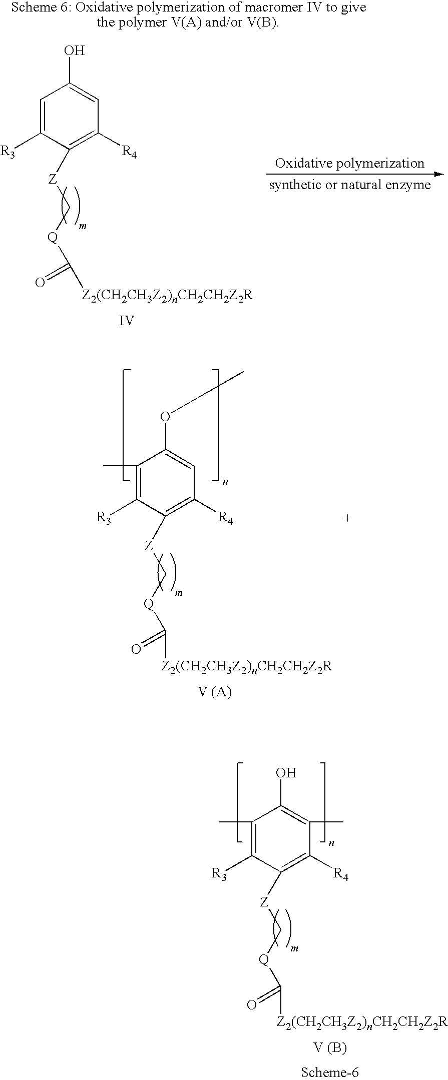

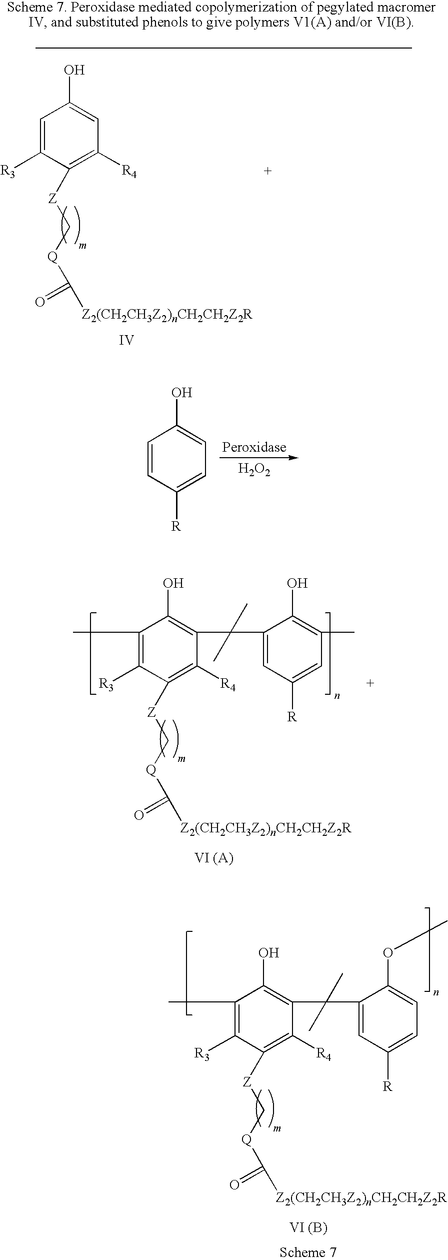

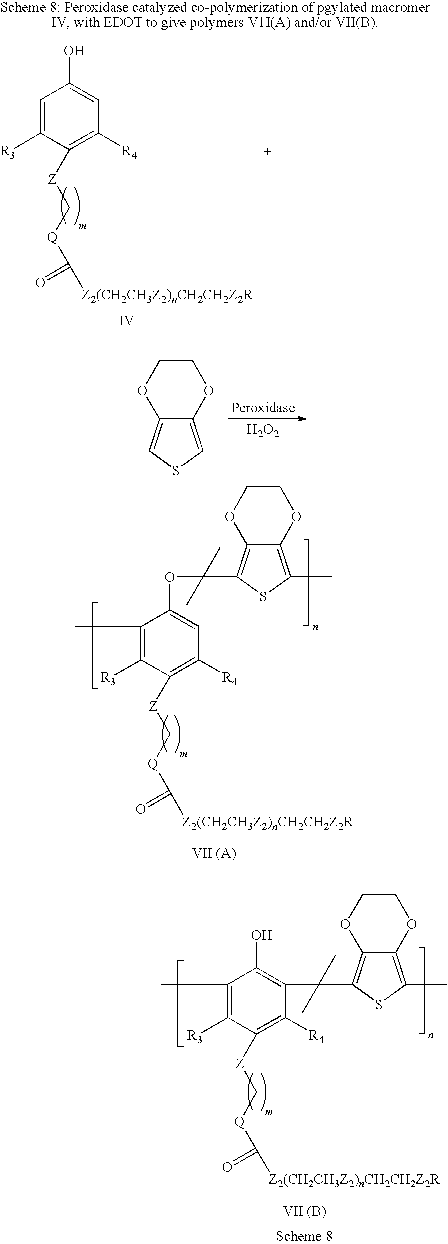

### Characterization of Polymers

The polymers obtained by the new methods can be characterized by known methods. For instance, the molecular weight and molecular weight distributions can be determined by gel permeation chromatography (GPC), matrix assisted laser desorption ionization (MALDI), and static or dynamic light scattering. Whereas the physical and thermal properties of the polymer products can be evaluated by thermal gravemetric analysis (TGA), differential scanning calorimetry (DSC), or surface tensiometer; the chemical structures of the polymers can be determined by, e.g., NMR (¹H, ¹³C NMR, ¹H-¹H correlation, or ¹H-¹³C correlation), IR, UV, Gas Chromatography-Electron Impact Mass Spectroscopy (GC-EIMS), EIMS, or Liquid Chromatography Mass Spectroscopy (LCMS).

### Applications and Uses of the Polymers

The new polymers I, II, III, V(A), V(B), VI(A), VI(B), VII(A) and/or VII(B), or mixtures thereof can have oligomeric poly(ethylene glycol) chains tethered to an irregularly arranged polymer main chain, thereby reducing the crystallization of poly(ethylene glycol). These polymers can provide very high free volume resulting in good segmental mobility while maintaining good mechanical properties, which results in very high ionic conductivity in polymer-ion complexes. Moreover, these polymer systems are inexpensive and are environmentally stable. The polymers and compositions containing the polymer are good candidates for opto-electronic applications such as polyelectrolytes in photovoltaic devices as well as in biosensor applications. These polymers have greater stability (thermal and mechanical stability) compared to polymer electrolytes synthesized from starting material having acidic functional groups (e.g., carboxylic acid) and are therefore of use in devices such as batteries and solar cells. Without being bound to theory it is believed that the greater stability of these electrolytes is due to the absence of acidic groups (e.g., carboxylic acid) in the preparation of these polymer electrolytes.

The stability of the fabricated devices using these polymeric electrolyte matrixes was tested at various temperatures ranging from room temperature to 90° C. by keeping them in an oven under controlled conditions. The photovoltaic performance of the devices was measured at predetermined intervals. The devices were found to be stable from 10 to 100 hours depending upon the temperature and polymer electrolyte.

These polymers can be used in the development of small, lightweight, powerful, safe, environmentally benign, and portable energy sources. These polymers can be used in batteries or solar cells that are small, light, easy to manufacture, and contain little or no toxic compounds.

These new polymers can also be used in dye-sensitized solar cell (DSSC)-devices based on molecular dyes. The DSSC devices are capable, in their photo-excited state, of injecting electrons into the conduction band of oxide semiconductors and have the potential to convert solar into electric energy at low cost. The polymer electrolytes also have several practical advantages compared to liquid electrolytes (leakage, desorption of the dye, corrosion of the electrodes) when used in DSSC devices. These polymers can be incorporated into redox electrolyte formulations and applied onto dye sensitized titanium oxide coated plastic substrates. After applying the electrolyte, the dye-sensitized titanium oxide coated flexible substrate can be sandwiched with a platinum coated indium tin oxide/polyester (ITO/PET) substrate and heat-sealed to obtain flexible plastic cells. The fabricated cells can be characterized for photovoltaic (PV) performance using a solar simulator at AM 1.5 conditions.

The presence of linkages such as ester and amide linkages in the polymers described herein as well as their high level of biocompatibility make these polymers good candidates for biomedical applications. For example, they can be used as biodegradable matrices for tissue engineering. These polymers can also be used to deliver therapeutic agents, such as a DNA, proteins, cells or drugs. Therapeutic agents can be used singularly, or in combination. Therapeutic agents can be, e.g., nonionic, or they may be anionic and/or cationic in nature. The amphiphilic nature of these polymers allow them to fold into specific conformations, e.g., to form micelles in aqueous solutions. Thus, they can be used to trap molecules such as drugs, e.g., camptothecin, etoposide, and other anticancer, antibiotic, antiviral, and related drug molecules, in aqueous media. The controlled delivery of the drug using these polymers can involve either one or a combination of diffusion, biodegradation, and osmosis. Drugs can be delivered by the diffusion or osmosis of the drug through the polymer matrix to a target organ or tissue.

The new polymers I, II, III, V(A), V(B), VI(A), VI(B), VII(A) and/or VII(B), or mixtures thereof can also undergo biodegradation through labile bonds (e.g., ester or amide) in which case the reactivity of the linkage becomes important. The drugs can be released in a controlled manner when the polymers are exposed to specific conditions, e.g., when the temperature or pH values change in the body at the target organ or tissue. This change in pH can cleave the linkages in the polymer, releasing the drug as the polymer is biodegraded in the body.

Alternatively, drugs can be chemically bonded to these polymers, e.g., via pendent amide, ester, or thioester bonds, which can further sustain the release of the drug. In such instances, biotransformation in the body, e.g., hydrolysis, liberates the active drug.

### EXAMPLES

The invention is further described in the following examples, which do not limit the scope of the invention described in the claims. Methods of making, analyzing, and characterizing some aspects of the polymer electrolytes are described below.

### Example 1

### Enzyme Catalyzed Synthesis of Functionalized Aromatic Polyesters

Generally, the aromatic polyesters are made by reacting the desired phenolic diester, such as dimethyl 5-hydroxyisophthalate, with the desired di-hydroxy terminated polyethylene glycol, such as 600 molecular weight polyethylene glycol, in the presence of NOVOZYME-435®. The reaction can take place in the presence of a solvent, but is conveniently run in the bulk at an elevated temperature, e.g., 65-95° C.

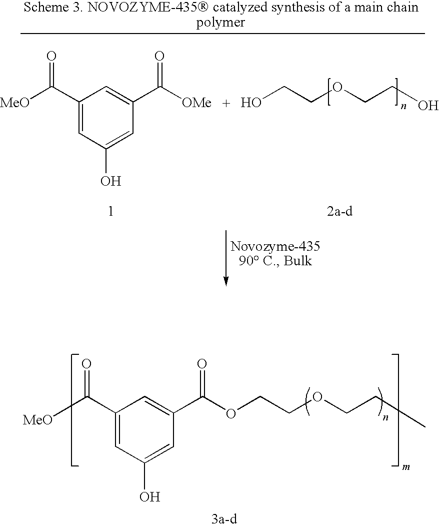

Dimethyl 5-hydroxyisophthalate (1, 1.0 mmol, 0.21 g) and polyethylene glycol (PEG) (1.0 mmol, M.W. 600 (0.6 g)(2a), 900 (0.9 g)(2b), and 1500 (1.5 g)(2c) and 300 (0.3 g)(2d)) were placed in a round-bottom flask (25 ml capacity). To this mixture was added NOVOZYME-435® (immobilized Candida antarctica lipase B), obtained from Novozymes, Denmark (10% by weight w.r.t. monomers, 0.80-1.7 g). The reaction flask was then placed in a constant temperature oil bath maintained at 90° C. under vacuum.

The reaction, as shown in Scheme 3, was allowed to proceed for 48 hours, after which the mixture was quenched by adding chloroform and filtering off the enzyme under vacuum. The organic solvent was then evaporated under vacuum and the residue was dialyzed using a membrane with a molecular weight cutoff of 6000. After the completion of dialysis, the product polymers 3a-3d (as described in further detail below) were freeze-dried.

In the polymerization without enzyme (control experiment), the monomers were recovered unchanged. Furthermore, no polymer formation was observed by using the deactivated Candida antarctica lipase B. These data imply that the present polymerizations proceeded through lipase catalysis.

The polymerization of 1 with PEG-300 (2d) under the same reaction conditions resulted in hardly any conversion to the copolymer (3d), probably because this PEG is of low molecular weight and its amount taken (in molar ratio to 1) is much less than that in the cases of 2a-2c.

The structures of the polymers were characterized using NMR spectroscopy (Bruker 500 MHz); and the molecular weights of the polymer products were determined by Gel Permeation Chromatography (GPC). The number average molecular weight of the polymers 3a-3c was found to be between 18000-23000 Da. The NMR results are indicated below.

### Poly[(poly(oxyethylene-600)-oxy-5-hydroxyisophlthaloyl](3a)

This polymer was obtained by heating dimethyl 5-hydroxyisophthalate (1 mmol, 0.21 g) with PEG 600 (1 mmol, 0.6 g) in the presence of NOVOZYME-435 (1 (0.8 g) at 90° C. in solvent free condition for 48 hours under vacuum 3a was obtained as a viscous oil after freeze-drying in 90% yield.

¹H NMR Data (CDCl₃): δ 3.64-3.68 (brs, methylene PEG protons on C-9 and C-10 carbons of the repeating units and on C-α and C-β), 3.82 (t, 2H, C-8H), 3.93 (s, 3H, —COOCH₃), 4.48 (t, 2H, C-7H), 7.71 (m, 2H, C-4H and C-6H) and 8.21 (s, 1H, C-2H).

¹³C NMR Data (CDCl₃): δ 52.74 (—OCH₃ end group), 62.07 (C-α), 64.74 (C-β, 69.44 (C-8), 70.93 (repeating PEG units' carbons), 72.90 (C-7), 121.43 (C-4 and C-6), 122.53 (C-2), 131.18 (C-1 and C-3), 157.57 (C-5) and 166.11 (—COOMe).

### Poly[(poly(oxyethylene-900)-oxy-5-hydroxyisophthaloyl] (3b)

This polymer was obtained by condensing dimethyl 5-hydroxyisophthalate (1 mmol, 0.21 g) with PEG 900 (1 mmol, 0.9 g) in presence of Novozyme-435 (1.1 g) at 90° C. in solvent free condition for 48 hours under vacuum. 3b was obtained as a waxy solid after freeze-drying in 93% yield.

¹H NMR Data (CDCl₃): δ 3.63-3.81 (brs, methylene PEG protons on C-9 and C-10 carbons of the repeating units and on C-α and C-β), 3.82 (t, 2H, C-8H), 3.92 (s, 3H, —COOCH₃), 4.46 (t, 2H, C-7H), 7.69 (d, 2H, C-4H and C-6H) and 8.73 (s, 1H, C-2H).

¹³C NMR Data (CDCl₃): δ 52.73 (—OCH₃ end group), 62.07 (C-α), 64.72 (C-β), 69.43 (C-8), 70.90 (repeating PEG units' carbons), 72.89 (C-7), 121.43 (C-4 and C-6), 122.51 (C-2), 131.99 (C-1 and C-3), 157.56 (C-5) and 166.38 (—COOMe).

### Poly[(poly(oxyethylene-1500)-oxy-5-hydroxyisophthaloyl] (3c)

This polymer was obtained by heating dimethyl 5-hydroxyisophthalate (1 mmol, 0.21 g) with PEG 1500 (1 mmol, 1.5 g) in the presence of Novozyme-435 (1.7 g) at 90° C. in solvent free condition for 48 hours under vacuum. 3c was obtained as a white solid after freeze-drying in 90% yield.

¹H NMR Data (CDCl₃): δ 3.6-3.79 (brs, methylene PEG protons on C-9 and C-10 carbons of the repeating units and on C-α and C-β), 3.86 (t, 2H, C-8H), 3.96 (s, 3H, —COOCH₃ end group), 4.51 (t, 2H, C-7H), 7.75 (s, 2H, C-4H and C-6H) and 8.24 (s, 1H, C-2H).

¹³C NMR Data (CDCl₃): δ 52.69 (—OCH₃ end group), 62.02 (C-α), 64.70 (C-β), 69.43 (C-8), 70.90 (repeating PEG units' carbons), 72.91 (C-7), 121.48 (C-4 and C-6), 122.38 (C-2), 131.95 (C-1 and C-3), 157.62 (C-5) and 166.07 (—COOMe).

### Example 2

### Synthesis of Heterofunctional PEG 4

Mono methoxy polyethylene glycol (PEG, Mwt 350, 1 mmol) and ethyl bromo acetate (1.2 nmol) were mixed together in round bottom flask and to this mixture was added Candida antarctica lipase B (immobilized on solid support, commonly known

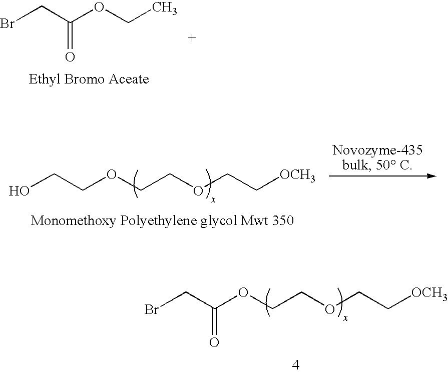

as NOVOZYME-435® (10% w/w wrt monomers). The reaction mixture was kept at 50° C. under nitrogen and monitored by thin layer chromatography (using a gradient solvent system of ethyl acetate in chloroform). After completion, the reaction was quenched by adding water and filtering off the enzyme. The aqueous filtrate so obtained was washed with hexane to remove any unreacted ethyl bromoacetate. The aqueous solution was freeze-dried to obtain the desired product, which was analyzed by its ¹H NMR and ¹³C NMR spectra.

¹H NMR (CDCl₃, 250 MHz): δ3.3-3.4 (s, 3H, OCH₃), 3.5-3.7 (PEG main chains protons), 3.85 (s, 2H, CH₂), 4.3 (t, 2H, COOCH₂).

### Example 3

### Coupling of Heterofunctional PEG 4 with Poly[(poly(oxyethylene)oxy-5-hydroxyisophthaloyl] (4a-4-c)

Equimolar quantities of 4a or 4b, 4c were dissolved in dry acetone, and to the resultant solution was added an equimolar amount of anhydrous potassium carbonate. The reaction mixture was refluxed, and progress of the reaction was monitored by TLC using ethyl acetate in petroleum ether (30%). After completion, the potassium carbonate was removed by filtration, and the filtrate so obtained was dried under vacuum by distilling off the acetone. The dried product was redissolved in water and dialyzed using (MWCO 1000) dialysis membrane. After the completion of dialysis, the product polymer

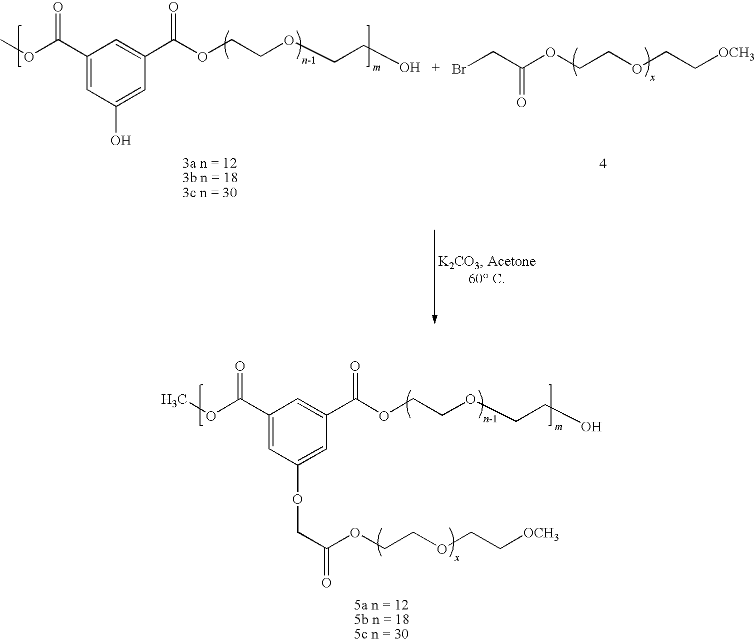

was obtained by freeze-drying the aqueous solution. The structure of the polymer was characterized by its spectral analysis.

### Poly(PEG 600-co-dimethyl 5-(-bromo ethyl methoxy peg 350)isophthalate), 5a

¹H NMR, (CDCl₃) δ 3.4 (s, 3H, OCH₃) 3.6-3.8 (bm, peg main chain and methoxy peg) 3.85 (t, 2H, —OCH₂—CH₂) 4.35 (t, 2H, COOH₂) 4.56 (t, 2H COOCH₂) 4.7 (s, 2H, OCH₂) 7.7 (2H, aromatic) 8.4 (1H, aromatic).

### Poly(PEG 900co-dimethyl 5-(-bromo ethyl methoxy peg 350)isophthalate), 5b

¹H NMR, (CDCl₃) δ3.3 (,3H, OCH₃) 3.5-3.9 (bm, peg main chain and methoxy peg) 4.35 (t, 2H, COOH₂) 4.5 (t, 2H COOCH₂) 4.7 (s, 2H, OCH₂) 7.7 (2H, aromatic) 8.4 (1H, aromatic).

### Poly(PEG 600-co-dimethyl 5-(bromo ethyl methoxy peg 350)isophthalate), 5c

¹H NMR, (CDCl₃) δ 3.3 (s, 3H, OCH₃) 3.5-3.8 (bm, peg main chain and methoxy peg) 3.89 (t, 2H, —OCH₂—CH₂) 4.3 (t, 2H, COOH₂) 4.56 (t, 2H COOCH₂) 4.7 (s, 2H, OCH₂) 7.7 (2H, aromatic) 8.4 (1H, aromatic)

### Example 4

### Coupling of Heterofunctional PEG 4 with Dihydroquinone

Equimolar amounts of dihydroquinone and heterofunctional PEG 4, were dissolved in dry acetone, and to the resultant solution was added an equimolar amount

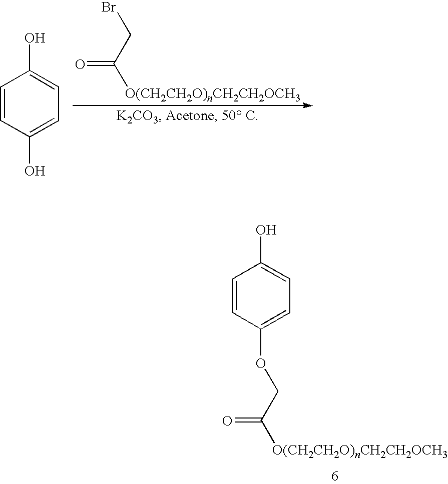

of anhydrous potassium carbonate. The reaction mixture was refluxed, and progress of the reaction was monitored by TLC using ethyl acetate in petroleum ether (30%). After completion, the potassium carbonate was removed by filtration, and the filtrate so obtained was dried under vacuum by distilling off the acetone. The dried product was re-dissolved in water and dialyzed using (MWCO 1000) dialysis membrane. After the completion of dialysis, the product polymer was obtained by freeze-drying the aqueous solution.

### Example 5

### General Method for Enzymatic Polymerizations

Generally, some second generation polymers are prepared by selecting the desired pegylated phenolic compound, mixing the phenolic compound with horseradish peroxidase in an aqueous buffered solution, such as sodium phosphate solution at pH 4-5, and then adding hydrogen peroxide to effect the polymerization.

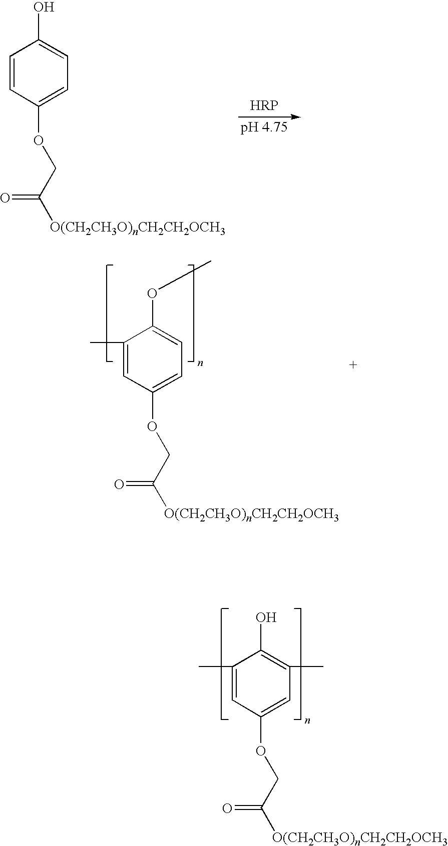

To a solution of pegylated phenol (0.070 mole) in sodium phosphate buffer (pH 4.75, 20 ml), 10 mg of the horseradish peroxidase (HRP) was added. The reaction vial was kept at 25° C. and the polymerization reaction was initiated by the addition of 4 ml of 0.03% hydrogen peroxide (added in small increment of 20 μl). The reaction was monitored by thin layer chromatography using a gradient solvent system of ethyl acetate in petroleum ether. After completion, the reaction was quenched by the addition of 5 ml of acetone and any precipitate obtained was filtered. The filtrate so obtained was concentrated and re-dissolved in water and dialyzed against water using a molecular weight cutoff of 1000. After dialysis, the solution was freeze-dried.

This enzymatic polymerization reaction was carried out with 3,4-ethylenedioxy-thiophene (EDOT, 0.035 mol) and macromer 6 (0.035 mol) to yield the product mixture of random polymers 8.

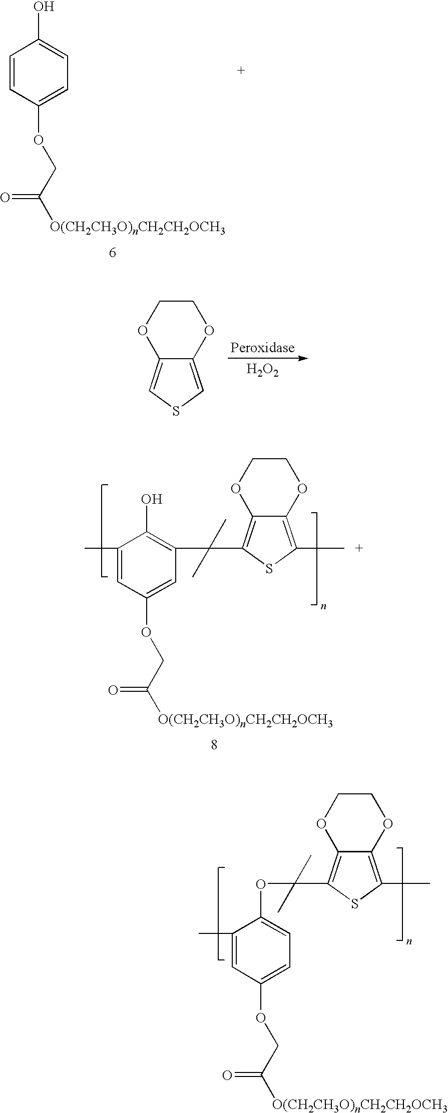

### Example 6

### Fabrication of Photoelectrochemical Cells and Photovoltaic Characterization

An aqueous suspension containing colloidal titanium dioxide nanoparticles was coated on either a SnO₂:F conducting oxide coated glass slide (sheet resistance of 15 Ω/cm²) or a flexible polyester substrate. The titanium dioxide coated glass slides were sintered at 120-450° C. for 30 minutes depending on the substrate. Titanium dioxide coating on ITO/polyester film was prepared using a low-temperature interconnection process. The treated titanium dioxide coatings on glass or flexible substrates were sensitized with Z907 [(ruthenium sensitizer cis-RuLL′(SCN)₂) (L=4,4′-dicarboxylic acid-2,2′-bipyridine, L′=4,4′-dinonyl-2,2′-bipyridine)] in an appropriate solvent. Quasi-solid electrolytes were prepared from the polymers described herein by adding 25-75 wt % of 2 M solution of 1,3-propylmethyl imidazolium iodide (ionic liquid) in butyrolactone.

Prepared electrolytes or electrolyte gels were applied onto the dye-sensitized titanium dioxide film and sandwiched with platinum-coated transparent conducting oxide glass or plastic substrate and sealed. The fabricated glass or flexible DSSCs were characterized using solar simulator under AM1.5 irradiation using a Xenon lamp with appropriate filters.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polymer of the formula: 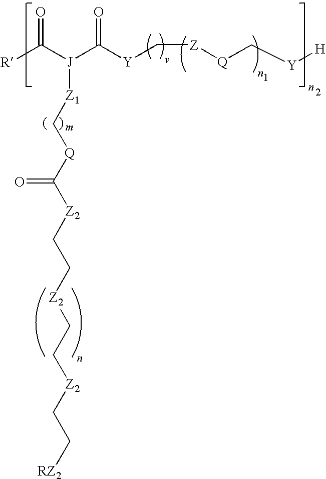
wherein
R is hydrogen, alkyl, aryl, or arylalkyl;
R′ is OH or alkoxy;
J is arylene, heteroarylene, or alkylene;
Y is independently O, S, or NH;
Z is O, S, or NH;
Z₁ is O, S, or NH;
Z₂ is independently O, S, or NH;
Q is alkylene, alkenylene, alkynylene, or arylene;
m is an integer of 0-100;
n is an integer of 1-1000;
n₁ is an integer of 1-1000;
n₂ is an integer of 1-10,000; and
v is 1, 2 or 3.

2. The polymer of claim 1, wherein v is 1 and m is 1.

3. The polymer of claim 1, wherein R is hydrogen.

4. The polymer of claim 1, wherein J is arylene.

5. The polymer of claim 4, wherein J is

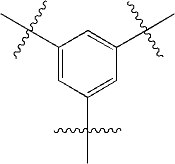

6. The polymer of claim 1, wherein Y is O and Z is O.

7. The polymer of claim 1, wherein Z₁ is O and Z₂ is O.

8. The polymer of claim 1, wherein Q is alkylene.

9. The polymer of claim 1, wherein the polymer has the formula: 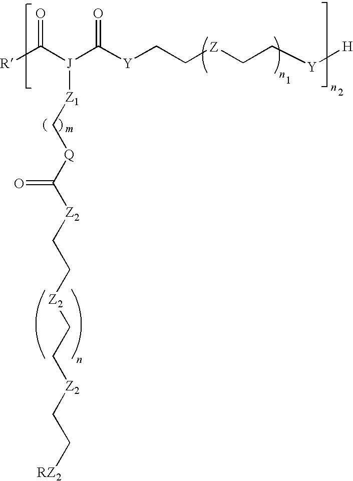
wherein
R is hydrogen, alkyl, aryl, or arylalkyl;
R′ is OH or alkoxy;
J is arylene, heteroarylene, or alkylene;
Y is independently O, S, or NH;
Z is O, S, or NH;
Z₁ is O, S, or NH;
Z₂ is independently O, S, or NH;
Q is allylene, alkenylene, alkynylene, or arylene;
m is an integer of 1-100;
n is an integer of 1-1000;
n₁ is an integer of 1-1000; and
n₂ is an integer of 1-10,000.

10. The polymer of claim 9, wherein R is hydrogen and R′ is OH.

11. The polymer of claim 9, wherein J is arylene.

12. The polymer of claim 11, wherein J is

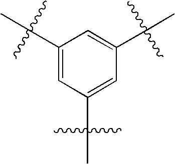

13. The polymer of claim 9, wherein Y is O and Z is O.

14. The polymer of claim 9, wherein Z₁ is O and Z₂ is O.

15. The polymer of claim 9, wherein Q is alkylene.

16. The polymer of claim 1, wherein the polymer has the formula: 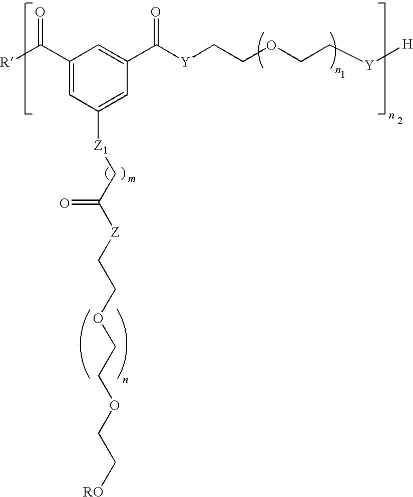
wherein
R is hydrogen, alkyl, aryl, or arylalkyl;
R′ is OH or alkoxy,
Y is independently O, S, or NH;
Z is O, S, or NH;
Z₁ is O, S, or NH;
m is an integer of 1-100;
n is an integer of 1-1000;
n₁ is an integer of 1-1000; and
n₂ is an integer of 1-10,000.

17. The polymer of claim 16, wherein R is hydrogen and R′ is OH.

18. The polymer of claim 16, wherein Y is O, Z is O, and Z₁ is O.

19. A polymer of the formula: 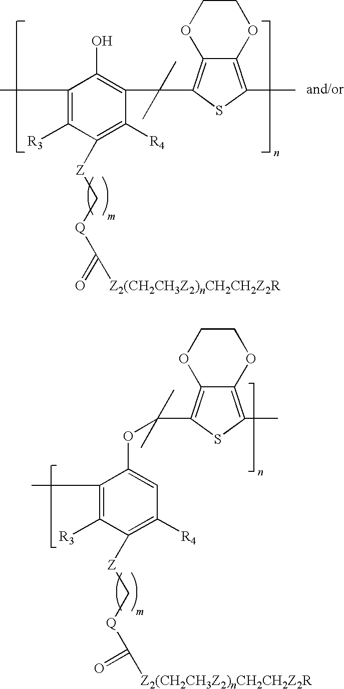
wherein R, R₃, and R₄ are independently hydrogen or alkyl; Z and Z₂ are independently O, S, or NH; Q is alkylene, alkenylene, alkynylene, or arylene; m is an integer of 1-100, and n is an integer of 1-10,000.

20. A polymeric mixture comprising: 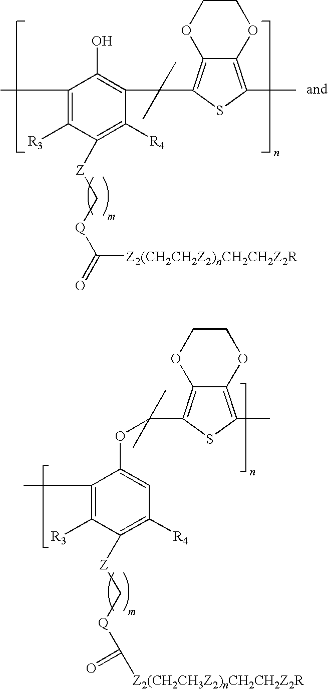
wherein R, R₃, and R₄ are independently hydrogen or alkyl; Z and Z₂ are independently O, S, or NH; Q is allylene, alkenylene, alkynylene, or arylene; m is an integer of 1-100; and n is an integer of 1-10,000.

21. The polymeric mixture of claim 20, wherein the mixture is a physical blend of the polymers.

22. A polymer of the formula: 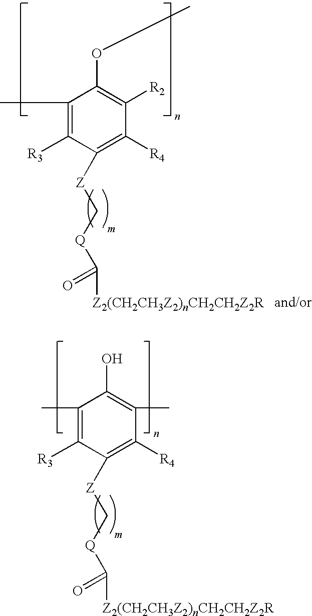
wherein R, R₂, R₃, and R₄ are independently hydrogen or alkyl; Z and Z₂ are independently O, S, or NH; Q is alkylene, alkenylene, alkynylene, or arylene; m is an integer of 1-100; and n is an integer of 1-10,000.

23. A polymeric mixture comprising: 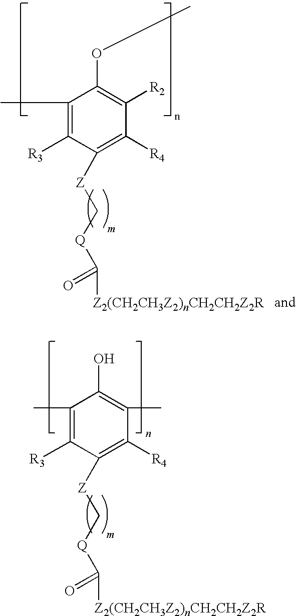
wherein R, R₂, R₃, and R₄ are independently hydrogen or alkyl; Z and Z₂ are independently O, S, or NH; Q is alkylene, alkenylene, alkynylene, or arylene; m is an integer of 1-100; and n is an integer of 1-10,000.

24. The polymeric mixture of claim 23, wherein the mixture is a physical blend of the polymers.

25. A polymer of the formula: 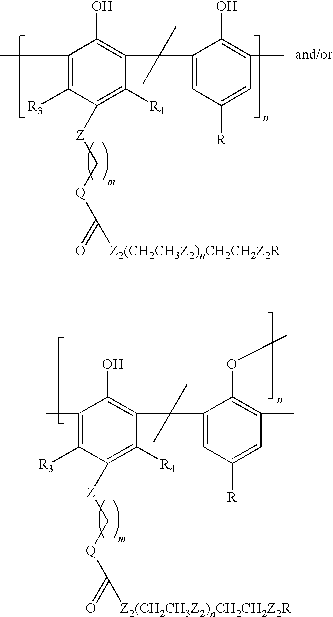
wherein R, R₃, and R₄ are independently hydrogen or alkyl; Z and Z₂ are independently O, S, or NH; Q is alkylene, alkenylene, alkynylene, or arylene; m is an integer of 1-100, and n is an integer of 1-10,000.

26. A polymeric mixture comprising: 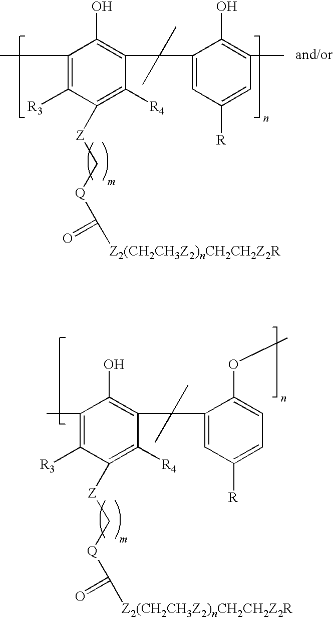
wherein R, R₃, and R₄ are independently hydrogen or alkyl; Z and Z₂ are independently O, S, or NH; Q is alkylene, alkenylene, alkynylene, or arylene; m is an integer of 1-100, and n is an integer of 1-10,000.

27. The polymeric mixture of claim 26, wherein the mixture is a physical blend of the polymers.

28. A composition comprising the polymer of claim 1.

29. A composition comprising the polymer of claim 1 and a therapeutic agent.

30. A composition comprising the polymer of claim 1 and an electrolyte, or a reaction product of the polymer of claim 1 and an electrolyte.

31. A battery comprising the composition of claim 30.

32. A solar cell comprising the composition of claim 30.

33. A macromer of the formula: 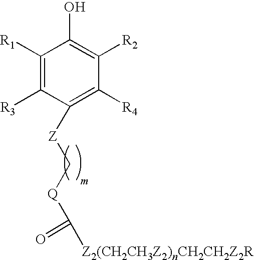
wherein R₁, R₂, R₃, and R₄ are independently hydrogen or alkyl; Z and Z₂ are independently O, S, or NH; Q is alkylene, alkenylene, alkynylene, or arylene; and
m is an integer of 1-100.

34. A method of preparing a macromonomer, the method comprising:
mixing a monomer or oligomer with an acyl compound to form a monomer mixture;
adding a lipase, an esterase, or a protease to the monomer mixture to form a reaction mixture;
reacting the reaction mixture for a time and under conditions suitable to yield a macromonomer, wherein the macromonomer is an alkylene ester, alkylene amide, or an alkylene thioester.

35. The method of claim 34, wherein the macromonomer is, 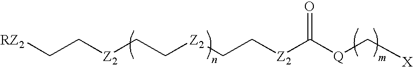
wherein
R is hydrogen, alkyl, aryl, or arylalkyl;
X is halide or sulfonate;
Q is alkylene, alkenylene, alkynylene, or arylene;
Z₂ is O, S, or NH;
m is an integer of 1-100; and
n is an integer of 1-1000.
